(19) United States
(12) Patent Application Publication
(10) Pub. No.: US 2016/0313309 A1
(43) Pub. Date: Oct. 27, 2016

(54) DIAGNOSTIC TEST STRIPS HAVING ONE OR MORE TEST PAD LAYERS AND METHOD OF USE THEREFORE

(71) Applicants: Ted Titmus, Mission Viejo, CA (US); William Pat Price, Henderson, NV (US)

(72) Inventors: Ted Titmus, Mission Viejo, CA (US); William Pat Price, Henderson, NV (US)

(21) Appl. No.: 14/694,930

(22) Filed: Apr. 23, 2015

(51) Int. Cl. G01N 33/52

(52) U.S. Cl. CPC G01N 33/525

(57) ABSTRACT

Some embodiments provide for a diagnostic test strip for detecting analytes having an active reference zone that contains a carrier strip and one or more test pads. Such test pads contain at least one, but preferably two transparent membranes or test pad layers. A first transparent membrane has a test reagent that indicates the presence of at least one reference analyte, while a second transparent membrane has a test reagent that indicates the presence of at least one target analyte. Test reagents are arranged on the membranes such that analyte detection produces a signal in any desired shape, such as a circle, oval, square, plus sign, an “X” sign, and/or a checkmark. Furthermore, a single membrane may contain two or more separate test reagents that detect the same or different analytes. Other embodiments provide for a method of detecting one or more analytes in a patient sample.

### BACKGROUND OF THE INVENTION

1. Field of the Invention

The invention generally relates to diagnostic assay materials. More specifically, the invention relates to diagnostic test strips having one or more test pads, each of which has one or more layers, and methods for the use of said diagnostic test strips.

2. Description of the Related Art

Many types of assays have been used to detect the presence of various substances, generally referred to as analytes, in physiological fluids such as urine and blood. These assays often involve antigen-antibody reactions; synthetic conjugates comprising radioactive, enzymatic, fluorescent, or visually observable metal sol tags; and specially designed reactor chambers. In all these assays, there is a receptor; e.g., an antibody or chemical, which is specific for the selected analyte; and a means for detecting the presence, and often the amount, of the analyte. While some tests are designed to make a quantitative determination, in many circumstances all that is required is a qualitative positive/negative indication. However, in some circumstances the analyte of interest is present in the test sample in very small concentrations. Such circumstances require an assay to be very sensitive in order to detect the presence, absence, and/or concentration of the desired analyte. False positives and false negatives for qualitative assays can also be especially problematic.

Unlike other forms of fluid specimens, the collection of oral fluid, such as saliva, for diagnostic purposes is complicated by many factors. These factors include the low volumes of salivary fluid secreted into the oral cavity, the relatively high viscosity of salivary fluid, and the diverse anatomic dispersion of the salivary glands. Moreover, many devices utilize surface tension, cohesion, adhesion, wicking, and/or capillary action to create lateral flow to contact the test sample with the test area. Such devices require substantial amounts of a liquid sample to provide lateral flow, yet many samples, such as saliva, have low and/or limited volumes. Because of these factors, the testing of salivary specimens has not been extensively developed. However, it is known that human saliva contains some of the same materials that may also be present in a human's blood. For example, it is known that human saliva carries lymphocytes, plasma cells and immunoglobulins that are directly related to the immunoglobulins found in the blood. In addition, saliva carries immunoglobulins that are believed to be peculiar to saliva, for example, the antibody known as secretory IgA. Because of the association between immunoglobulins of the blood and saliva, as well as the occurrence of secretory IgA, antigen-antibody tests may prove useful for conducting diagnostic assays with saliva, amongst other forms of physiological fluids.

Many technological fields and applications require rapid, accurate, and reproducible analyte detection. This is especially true for law enforcement officials, physicians, aid workers, employers, parents, and other assay users because safety and livelihood could depend upon the presence, absence, and/or concentration of certain analytes. In some circumstances, untrained individuals may need to test for the presence of an analyte to determine personal safety and health. However, common techniques for collecting and testing specimens are often complicated or invasive. For example, collecting salivary fluid often involves the use of capillary tubes, micropipette suctioning, chewing on paraffin or foam, and/or aspiration from the mouth into polypropylene syringes. Such techniques inevitably complicate the collection of salivary specimens and increase the likelihood of user error and/or false negatives. What is needed is a simple, accurate assay that provides trustworthy signaling of the presence, absence, and/or concentration of one or more analytes in a given sample. These and other objects and features of the invention will be apparent from the following description, drawings, and claims.

### SUMMARY OF THE INVENTION

Some embodiments provide for a diagnostic test strip for detecting analytes having an active reference zone that contains a carrier strip and at least one test pad. Such test pads contain at least one, but preferably at least two transparent membranes as test pad layers. A first transparent membrane has a test reagent that indicates the presence of at least one reference analyte while a second transparent membrane has a test reagent that indicates the presence of at least one target analyte. The test reagents are arranged on the membranes in a substantially single striped shape, and the transparent membranes are opposed to each other such that the striped shapes are at substantially right angles with at least one test pad in fluid contact with the carrier strip. In other embodiments, the test reagents are arranged in a substantially single striped shape on a portion of the transparent membranes, and the transparent membranes are opposed to each other such that detecting both the reference analyte and the target analyte produces a signal in various shapes, such as a circle, oval, square, plus sign, an “X” sign, and/or a checkmark. Optionally, transparent membranes may be opposed with test reagents such that the detection of both a target analyte and a reference analyte produces a signal within a signal. Furthermore, a single membrane may contain two or more separate test reagents for target analytes, each reagent disposed at substantially right angles to any test reagent for the reference analyte on another membrane.

Some embodiments may have a single test pad, while other embodiments may have two or more test pads. A test pad may detect the same, or optionally different, target analytes. Moreover, two or more test pads may detect different markers on the same analyte. Two or more test pads may touch each other on the carrier strip, or two or more test pads may optionally be separated on the carrier strip. Moreover, two of the two or more test pads may be on opposite sides of the carrier strip. Any test pad may be substantially covered in an oxygen-impermeable water-soluble membrane. Furthermore, one or more test pads may be in direct fluid contact with each other, and test pads may be in fluid contact with the carrier strip. In still other embodiments, the test strip may comprise an element that mechanically fixes the test pad to the carrier strip

In some embodiments, the reference analyte and the target analyte are optionally found in patient samples such as saliva, sputum, blood serum, blood plasma, blood, urine, semen, ascites, cerebral spinal fluid, and/or fecal matter. The reference analyte may be alpha-amylase while the target analyte may optionally be any one or more drugs of abuse and/or therapeutic drugs.

Other embodiments provide for a method of detecting one or more analytes in a patient sample by contacting one or more test pads of an embodiment of a diagnostic test strip with a patient sample and reading the results from the embodiment. Patient samples may optionally be serum, semen, urine, saliva, blood, ascites, sputum, cerebral spinal fluid, and/or fecal material. Moreover, embodiments may be directly contacted with a patient's urine stream, source of bleeding, and/or tongue. Optionally, signaling reagents may be applied to one or more test pads of an embodiment of a diagnostic test strip.

### BRIEF DESCRIPTION OF THE DRAWINGS

FIGS. 1A-1C are a top view of an embodiment of a diagnostic test strip having a square test pad comprising two layers such that it is capable of producing a signal in the form of a checkmark.

FIG. 1D is a perspective view of an embodiment of a diagnostic test strip having a square test pad comprising two layers such that it is capable of producing a signal in the form of a checkmark.

FIGS. 1E and 1F are perspective views of an embodiment of a test pad.

FIGS. 2A-2C are a top view of an embodiment of a diagnostic test strip having a circular test pad comprising two layers such that it is capable of producing a signal in the form of a “plus” sign.

FIG. 2D is a perspective view of an embodiment of a diagnostic test strip having a circular test pad comprising two layers such that it is capable of producing a signal in the form of a “plus” sign.

FIG. 2E is an expanded view of an embodiment of a circular test pad having two test pad layers.

FIG. 2F is a perspective view of an embodiment of a circular test pad having two test pad layers.

FIG. 2G is a top view of an embodiment of a diagnostic test strip having a circular test pad comprising four layers such that it is capable of producing a signal in the form of a “plus” sign.

FIG. 2H is a perspective view of an embodiment of a diagnostic test strip having a circular test pad comprising four layers such that it is capable of producing a signal in the form of a “plus” sign.

FIG. 2I is an expanded view of an embodiment of a circular test pad having four test pad layers.

FIG. 2J is a perspective view of an embodiment of a circular test pad having two four pad layers.

FIG. 2K is a top view of an embodiment of a diagnostic test strip having two circular test pads, one comprising two layers and the other comprising four layers, such that it is capable of producing two signal in the form of a “plus” sign.

FIG. 2L is a perspective view of an embodiment of a diagnostic test strip having two circular test pads, one comprising two layers and the other comprising four layers, such that it is capable of producing two signal in the form of a “plus” sign.

FIG. 3A is a top view of an embodiment of a diagnostic test strip having a square test pad comprising two layers such that it is capable of producing a signal in the form of a “plus” sign.

FIG. 3B is a perspective view of an embodiment of a diagnostic test strip having a square test pad comprising two layers such that it is capable of producing a signal in the form of a “plus” sign.

FIG. 3C is an expanded view of an embodiment of a square test pad having two test pad layers.

FIG. 3D is a perspective view of an embodiment of a square test pad having two test pad layers.

FIG. 3E is an expanded view of an embodiment of a square test pad having four test pad layers.

FIG. 3F is a perspective view of an embodiment of a square test pad having two four pad layers.

FIG. 4A is a top view of an embodiment of a diagnostic test strip having three square test pads comprising two layers such that it is capable of producing a signal in the form of a “plus” sign in each of the three test pads.

FIG. 4B is a perspective view of an embodiment of a diagnostic test strip having three square test pads comprising two layers such that it is capable of producing a signal in the form of a “plus” sign in each of the three test pads.

FIG. 5A is a top view of an embodiment of a diagnostic test strip having one rectangular test pad comprising two layers such that it is capable of producing three signals in the form of a “plus” sign.

FIG. 5B is a perspective view of an embodiment of a diagnostic test strip having one rectangular test pad comprising two layers such that it is capable of producing three signals in the form of a “plus” sign.

FIG. 6A is a top view of an embodiment of a diagnostic test strip having one rectangular test pad comprising four layers such that it is capable of producing three signals in the form of a “plus” sign.

FIG. 6B is a perspective view of an embodiment of a diagnostic test strip having one rectangular test pad comprising four layers such that it is capable of producing three signals in the form of a “plus” sign.

### DETAILED DESCRIPTION

The present application relates to U.S. patent application Ser. No. ______, filed ______ entitled “DIAGNOSTIC TEST STRIPS WITH MULTIPLE LAMINATED LAYERS CONTAINING ONE OR MORE REAGENT-CARRYING PADS IN ONE OR MORE LAYERS”, Attorney Docket Number TTUSA.005A2, U.S. patent application Ser. No. ______, filed ______ entitled “MECHANICAL ATTACHMENT OF TEST PADS TO A DIAGNOSTIC TEST STRIP”, Attorney Docket Number TTUSA.006A2, U.S. patent application Ser. No. ______, filed ______ entitled “MECHANICAL ATTACHMENT OF TEST PADS TO A DIAGNOSTIC TEST DEVICE”, Attorney Docket Number TTUSA.007A2, U.S. patent application Ser. No. ______, filed ______ entitled “DIAGNOSTIC TEST STRIP WITH SELF-ATTACHING TEST PADS AND METHODS OF USE THEREFORE”, Attorney Docket Number TTUSA.008A2, U.S. patent application Ser. No. ______, filed ______ entitled “DIAGNOSTIC TEST STRIPS WITH FLASH MEMORY DEVICES AND METHODS OF USE THEREFORE”, Attorney Docket Number TTUSA.009A2, U.S. patent application Ser. No. ______, filed ______ entitled “DIAGNOSTIC TEST STRIP FOR ORAL SAMPLES AND METHOD OF USE THEREFORE”, Attorney Docket Number TTUSA.010A2, U.S. patent application Ser. No. ______, filed ______ entitled “SINGLE USE MEDICAL TEST PACKAGING”, Attorney Docket Number TTUSA.012A2, U.S. patent application Ser. No. ______, filed ______ entitled “DIAGNOSTIC TEST STRIPS FOR DETECTION OF PAST OR PRESENT INFECTION OF VARIOUS STRAINS OF HEPATITIS” Attorney Docket Number TTUSA.013A2, and U.S. patent application Ser. No. ______, filed ______ entitled “DIAGNOSTIC TEST STRIPS FOR DETECTION OF PRE-SPECIFIED BLOOD ALCOHOL LEVEL” Attorney Docket Number TTUSA.014A2, all of whom have the inventors Ted Titmus and William Pat Price, all of which are filed herewith this even date, all of the disclosures of which are hereby expressly incorporated by reference in their entirety and are hereby expressly made a portion of this application.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. Descriptions of unnecessary parts or elements may be omitted for clarity and conciseness, and like reference numerals refer to like elements throughout. In the drawings, the size and thickness of layers and regions may be exaggerated for clarity and convenience. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Some embodiments of the invention provide for a diagnostic test strip having an active reference zone containing a carrier strip and one or more test pads having one or more transparent membranes that contain test reagents and/or signaling reagents that detect analytes. Described in more detail below, analytes may be reference analytes, or they may be target analytes. The one or more test pads are optionally located on any side of the carrier strip, including being located on the opposite and/or same side of the carrier strip.

Other embodiments provide for a method of detecting one or more analytes in a patient sample by contacting one or more test pads of an embodiment of a diagnostic test strip with a patient sample and reading the results from the embodiment. Moreover, embodiments may be directly contacted with a patient's sample or the source of the sample. These methods include contacting the test strip with one or more signaling reagents so that the one or more reagents contact the one or more test pads.

Any method's results may be read visually by an embodiment's user, if the application so desires, and/or any method's results may be stored in a memory device for recordation and later access. Alternatively, the results may be read by someone other than the user or the supplier of the sample. In some circumstances, the results of the method will be restricted from the user of the embodiment and/or the supplier of the sample analyzed.

Embodiments of the invention can be used to detect any analyte which has heretofore been assayed using known immunoassay procedures, or known to be detectable by such procedures. Furthermore, it is envisioned that known methods can be modified as needed to afford suitable test reagents and/or signaling reagents that will detect analytes that are similar to analytes that have been previously detected using known procedures.

As disclosed below, various features of the embodiments and methods of using the embodiments enable both trained and untrained personnel to reliably detect the presence, absence, and/or concentration of one or more analytes in a sample. Indeed, features of the embodiments and methods for their use allow for the detection of extremely small quantities of one or more particular analytes while avoiding false positives and false negatives. Furthermore, features of the embodiments and methods for their use allow for accurate and trustworthy attainment and/or storage of information related to the tested sample. Optionally, embodiments may both produce a signal that communicates information to the user and/or store information related to the test sample in one or more memory devices. Consequently, the invention is ideal for use in both prescription and over-the-counter assay test kits which will enable a consumer to self diagnose themselves and others, or test food and/or water prior to consumption.

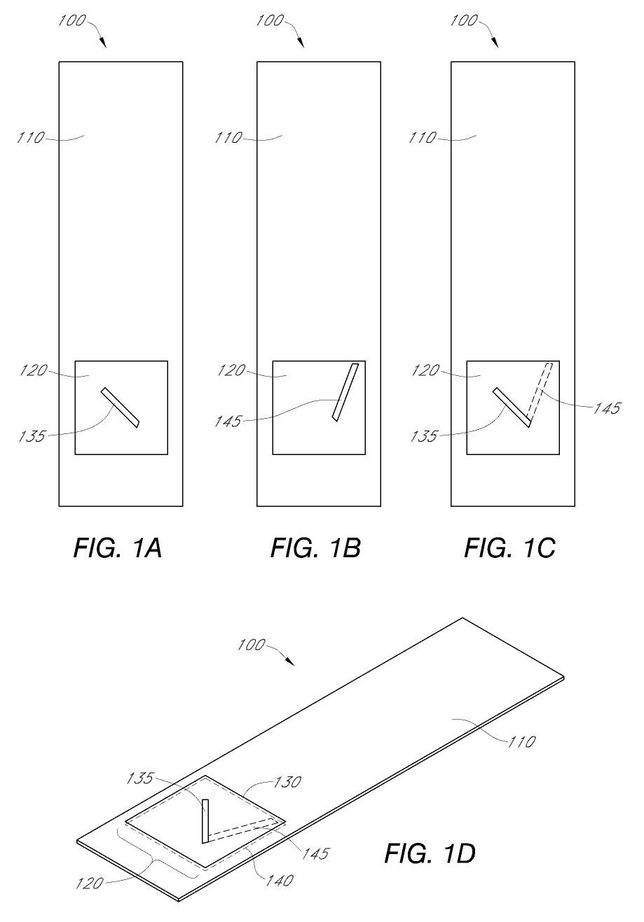

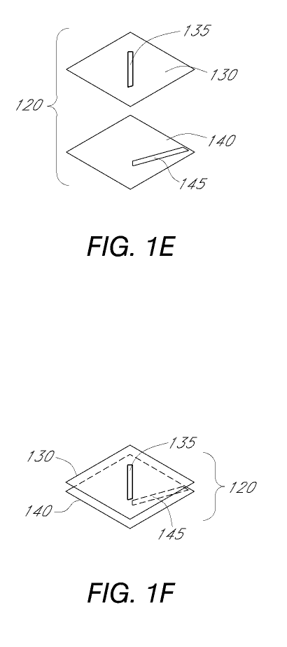

Referring to the drawings, FIGS. 1A-1C illustrate schematically a top view of an embodiment of a diagnostic test strip, 100, having a carrier strip, 110, and one square test pad, 120, located on carrier strip 110. In FIG. 1A, test pad 120 possesses a signal, 135, resulting from the detection of an analyte by a test reagent and/or a signaling reagent. Likewise, in FIG. 1B, test pad 120 possesses a signal, 145, resulting from the detection of an analyte by a test reagent and/or a signaling reagent. Optionally, signals 135 and 145 may result from detection of the same analyte, different analytes, and/or different markers for the same analyte. In FIG. 1C, test pad 120 demonstrates that upon detection of one or more analytes or markers of analytes such that signals 135 and 145 are generated, a “checkmark” will appear to the user.

FIG. 1D illustrates schematically a perspective view of diagnostic test strip 100. In FIG. 1D, test pad 120 is shown as comprising two test pad layers, 130 and 140. Test pad layers 130 and 140 generate signals 135 and 145, respectively, upon detection of one or more analytes or markers of analytes. FIGS. 1E and 1F further illustrate test pad 120, test pad layers 130 and 140, and signals 135 and 145.

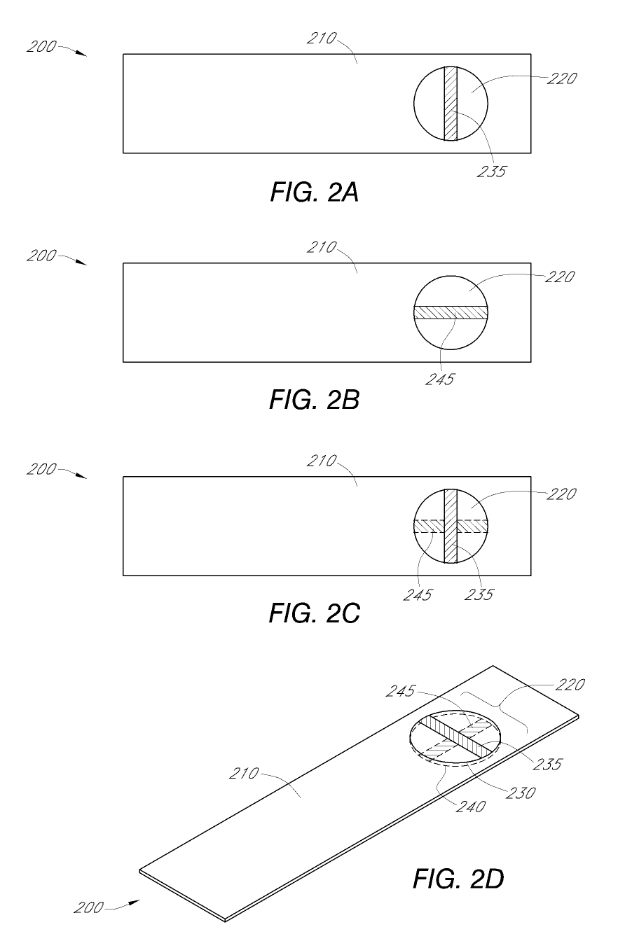

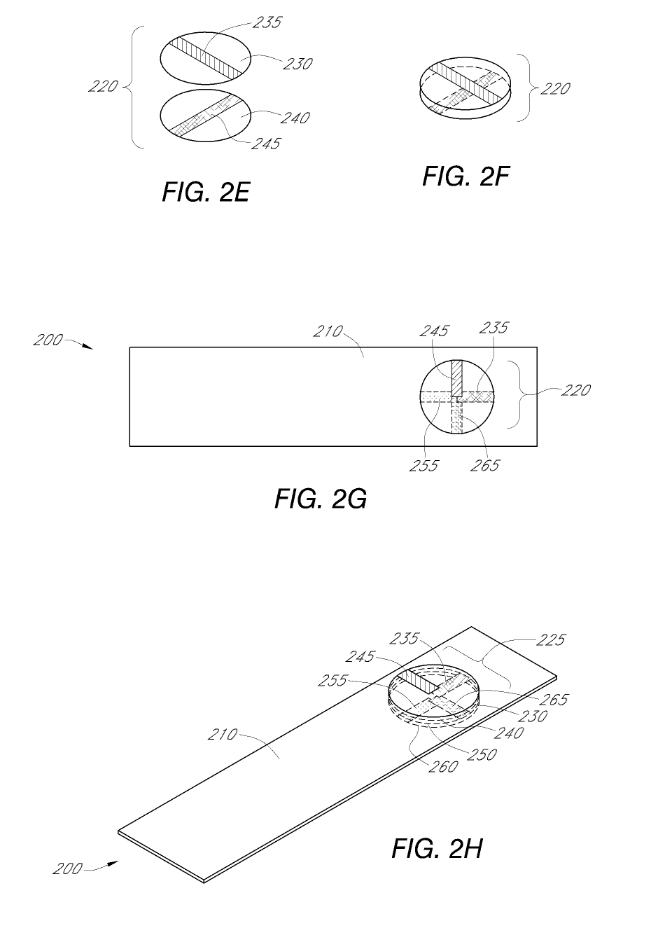

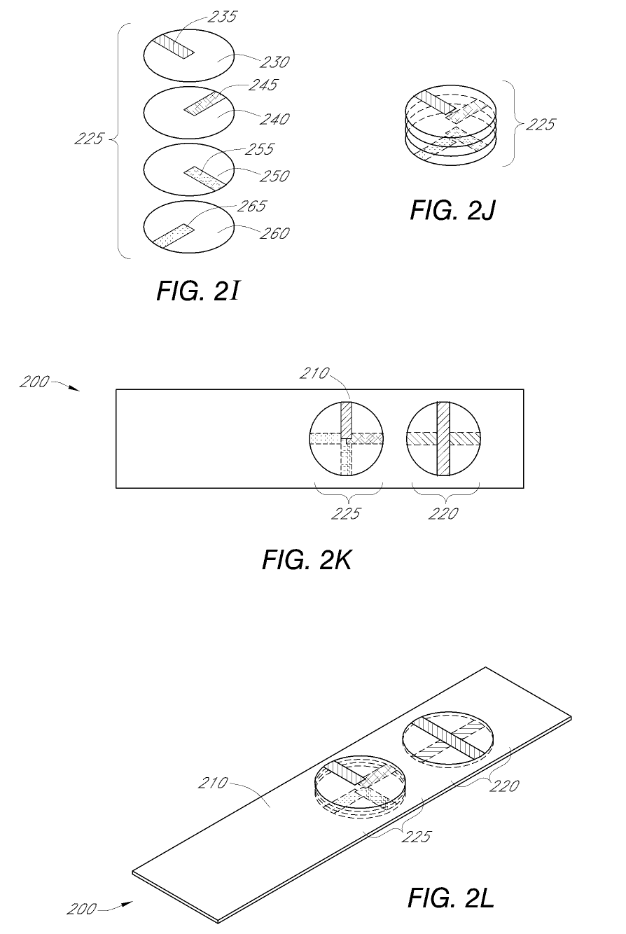

FIGS. 2A-2C illustrate schematically a top view of an embodiment of a diagnostic test strip, 200, having a carrier strip, 210, and one circular test pad, 220, located on carrier strip 210. In FIG. 2A, test pad 220 possesses a signal, 235, resulting from the detection of an analyte by a test reagent and/or a signaling reagent. Likewise, in FIG. 2B, test pad 220 possesses a signal, 245, resulting from the detection of an analyte by a test reagent and/or a signaling reagent. Optionally, signals 235 and 245 may result from detection of the same analyte, different analytes, and/or different markers for the same analyte. In FIG. 2C, test pad 220 demonstrates that upon detection of one or more analytes or markers of analytes such that signals 235 and 245 are generated, a “plus” sign will appear to the user.

FIG. 2D illustrates schematically a perspective view of diagnostic test strip 200. In FIG. 2D, test pad 220 is shown as comprising two test pad layers, 230 and 240. Test pad layers 230 and 240 generate signals 235 and 245, respectively, upon detection of one or more analytes and/or markers of analytes. FIGS. 2E and 2F further illustrate test pad 220, test pad layers 230 and 240, and signals 235 and 245. FIGS. 2E and 2F illustrate how layers 230 and 240 can be stacked such that signals 235 and 245 can produce a “plus” sign.

FIG. 2G is a top view of an embodiment of a diagnostic test strip, 200, having a carrier strip, 210, and one circular test pad, 225, located on carrier strip 210. In FIG. 2G, test pad 225 demonstrates the production of four signals, 235, 245, 255, and 265, resulting from the detection of one or more analytes or markers of analytes. FIG. 2H is a perspective view of the embodiment schematically illustrated in FIG. 2F. In FIG. 2H, test pad 225 is shown as comprising four test pad layers, 230, 240, 250, and 260. Test pad layers 230, 240, 250, and 260 generate signals 235, 245, 255, and 265, respectively, upon detection of one or more analytes or markers of analytes. FIGS. 2I and 2J further illustrate test pad 225, test pad layers 230, 240, 250, and 260, and signals 235, 245, 255, and 265. FIGS. 2I and 2J illustrate how layers 230, 240, 250, and 260 can be stacked such that signals 235, 245, 255, and 265 can produce a “plus” sign.

FIG. 2K illustrates schematically a top view of an embodiment of a diagnostic test strip, 200, having a carrier strip, 210, and two circular test pads, 220 and 225, located on carrier strip 210. Similarly, FIG. 2L illustrates schematically a perspective view of an embodiment of a diagnostic test strip, 200, having a carrier strip, 210, and two test pads, 220 and 225, located on carrier strip 210. Optionally, diagnostic test strips may possess multiple test pads, each comprising multiple test pad layers. Moreover, any shape of test pad may be incorporated into an embodiment.

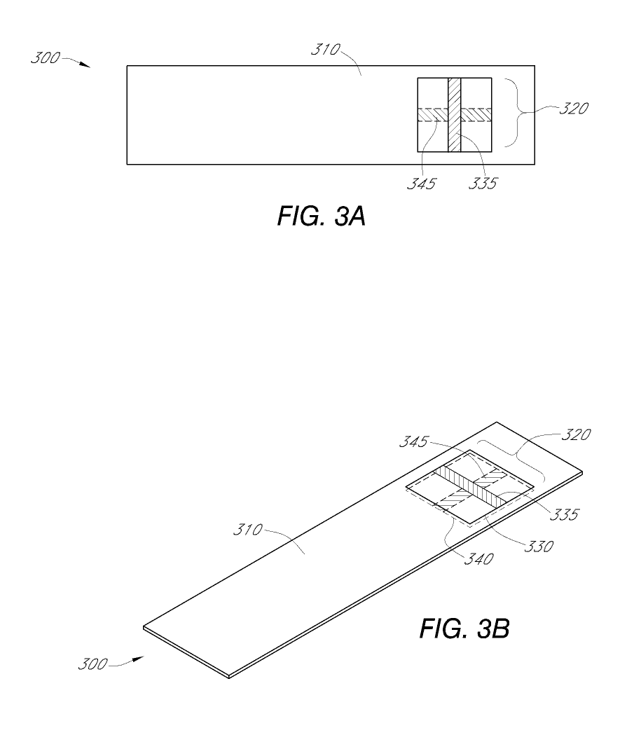

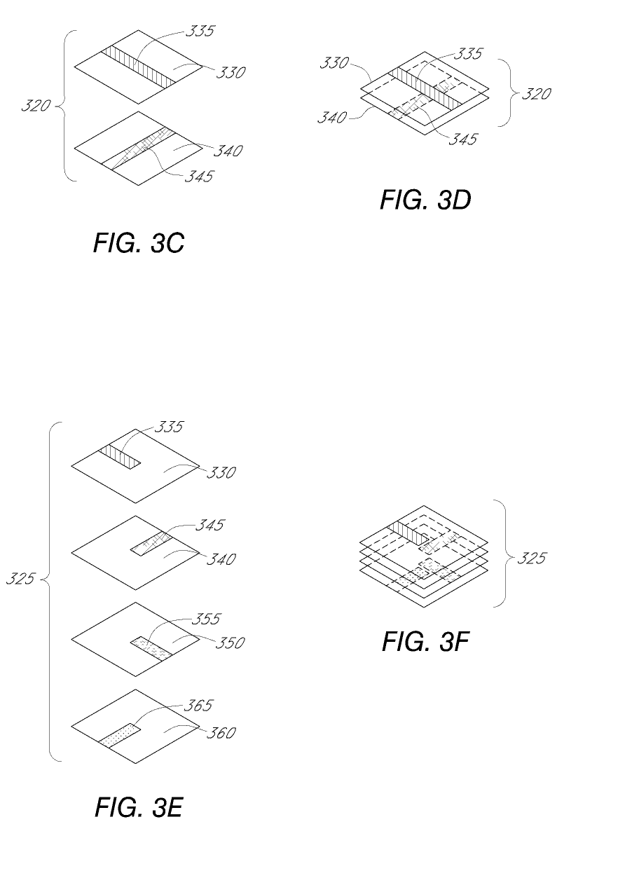

FIG. 3A illustrates schematically a top view of an embodiment of a diagnostic test strip, 300, having a carrier strip, 310, and one square test pad, 320, located on carrier strip 310. In FIG. 3A, test pad 320 possesses two signals, 335 and 345, resulting from the detection of an analyte by a test reagent and/or a signaling reagent. Optionally, signals 335 and 345 may result from detection of the same analyte, different analytes, and/or different markers for the same analyte. In FIG. 3A, test pad 320 demonstrates that upon detection of one or more analytes or markers of analytes such that signals 335 and 345 are generated, a “plus” sign will appear to the user.

FIG. 3B illustrates schematically a perspective view of diagnostic test strip 300. In FIG. 3B, test pad 320 is shown as comprising two test pad layers, 330 and 340. Test pad layers 330 and 340 generate signals 335 and 345, respectively, upon detection of one or more analytes or markers of analytes. FIGS. 3C and 3D further illustrate test pad 320, test pad layers 330 and 340, and signals 335 and 345. FIGS. 3C and 3D illustrate how layers 330 and 340 can be stacked such that signals 335 and 345 can produce a “plus” sign. Optionally, a test pad may comprise four test pad layers, as illustrated in FIGS. 3E and 3F for test pad 325. Test pad 325 comprises test pad layers 330, 340, 350, and 360, each capable of generating a signals 335, 345, 355, and 365, respectively. FIGS. 3E and 3F illustrate how layers 330, 340, 350, and 360 can be stacked such that signals 335, 345, 355, and 365 can produce a “plus” sign.

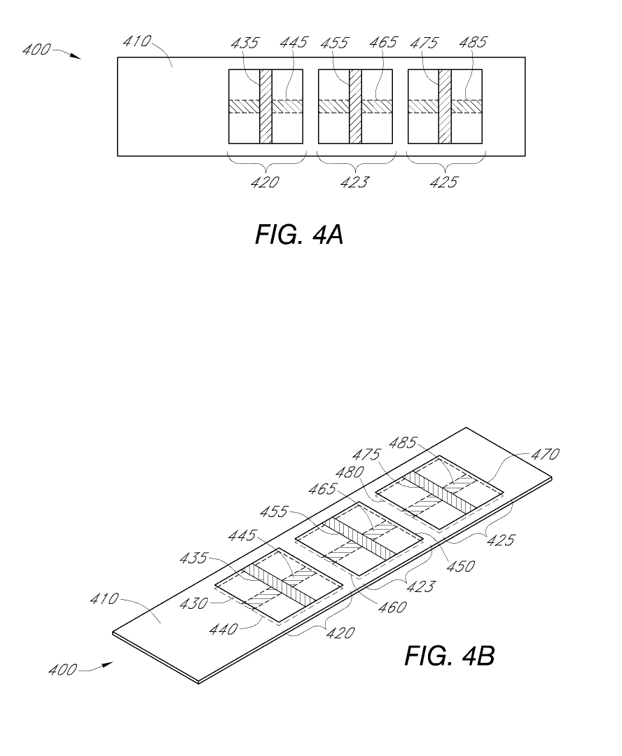

FIGS. 4A and 4B illustrate an embodiment of a diagnostic test strip, 400, having a carrier strip, 410, and three square test pads, 420, 423, and 425, located on carrier strip 410. In FIG. 4A, representing 400 from a top view, test pads 420, 423, and 425 are illustrated as displaying signals 435 and 445 on test pad 420, signals 455 and 465 on test pad 423, and signals 475 and 485 on test pad 425. Optionally, signals 435, 445, 455, 465, 475, and 485 may result from detection of the same analyte, different analytes, and/or different markers for the same analyte. In FIG. 4B, diagnostic test strip 400 is illustrated schematically in a perspective view. In this view, one can see that test pads 420, 423, and 425 comprise two test pad layers, 430 and 440, 450 and 460, and 470 and 480, respectively. Signals 435 and 445 are produced in layers 430 and 440, respectively, signals 455 and 465 are produced in layers 450 and 460, respectively, and signals 475 and 485 are produced in layers 470 and 480, respectively. In FIGS. 4A and 4B, test pads 420, 423, and 425 demonstrate that upon detection of one or more analytes or markers of analytes such that signals 435, 445, 455, 465, 475, and 485 are generated, “plus” signs will appear to the user in each test pad.

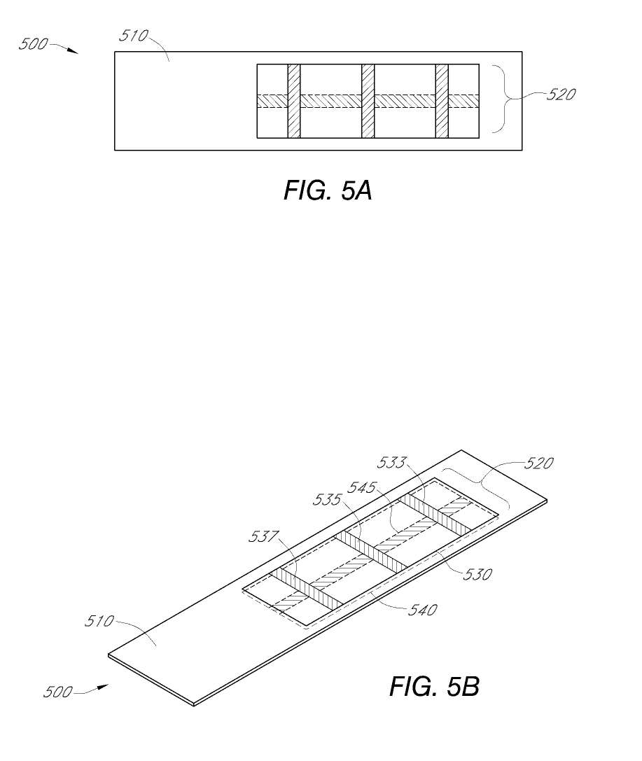

FIGS. 5A and 5B illustrate an embodiment of a diagnostic test strip, 500, having a carrier strip, 510, and one rectangular test pad, 520, located on carrier strip 510. As FIG. 5A, represents, however, test pad 520 is capable of detecting anywhere from one to four analytes to generate signals 533, 535, 537, and 545. Optionally, signals 533, 535, 537, and 545 may result from detection of the same analyte, different analytes, and/or different markers for the same analyte. In FIG. 5B, diagnostic test strip 500 is illustrated schematically in a perspective view. In this view, one can see that test pad 520 comprises two test pad layers, 530 and 540. Signals 533, 535, and 537 are produced in layer 530, respectively, while signal 545 is produced in layer 540. In FIGS. 4A and 4B, test pad 520 demonstrate that upon detection of one or more analytes or markers of analytes such that signals 533, 535, 537, and 545 are generated, one to three “plus” signs will appear to the user.

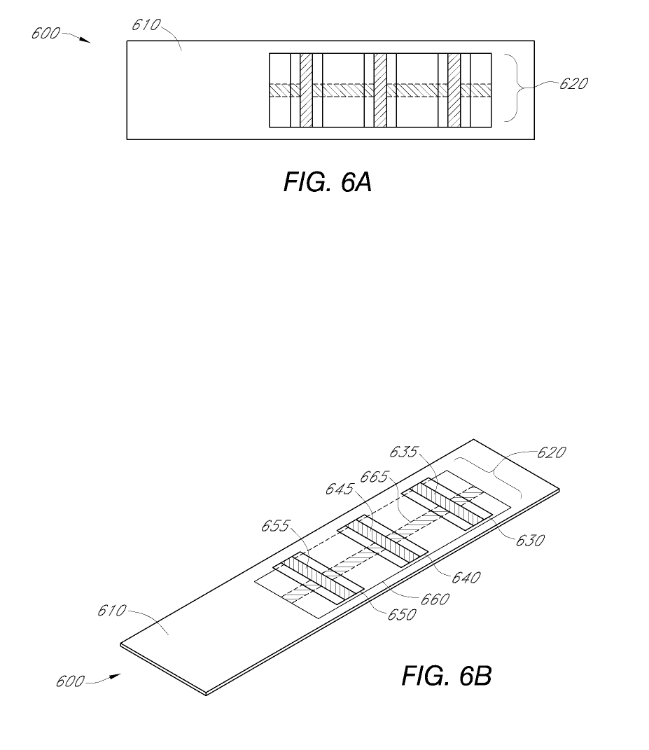

FIGS. 6A and 6B illustrate an alternative embodiment of a diagnostic test strip, 600, that functions similarly to embodiment 500. FIG. 6A illustrates a top view of a diagnostic test strip, 600, having a carrier strip, 610, and one rectangular test pad, 620, located on carrier strip 610. Test pad 620 is illustrated as comprising four test pad layers, 630, 640, 650, and 660, of which layers 630, 640, 650 cover portions of test pad layer 660. Consequently, analyte detection by test pad 620 can result in the production of one, two, and/or three “plus” signs resulting from signals 635, 645, 655, and 665. Test pad layers 630, 640, 650, and 660 are capable of generating signals 635, 645, 655, and 665, respectively, upon detection of the same analyte, different analytes, and/or different markers for the same analyte. Thus, test pad 620 is capable of detecting anywhere from one to four analytes and/or markers of analytes. FIG. 6B illustrates test pad 600 in a perspective view and further demonstrates the multiple layer composition of test pad 620.

### Carrier Strip

The carrier strip provides structural support for the one or more test pads and the one or more boundary projections. As a structural support, many materials suitable for use in preparing the carrier strip are known in the art. Such materials include but are not limited to plastics including polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch material, polyvinylchloride, nylon, and other polyamides, metals, alloys, ceramics, glass, wood, cardboard, paper, natural rubber, synthetic rubber, and other suitable polymers. Optionally, the carrier strip may be porous or non-porous. Optionally, the carrier strip may facilitate the transmission of information from the one or more test pads to a memory device. Transmitted information may include, but is not limited to, the presence, absence, and/or concentration of one or more analytes of interest. The carrier strip may facilitate the transmission of information from the one or more test pads to the one or more memory devices by any of several methods known in the art. Such methods include, but are not limited to, the transmission of electrical signals which result from changes in the coulometry, amperometry, or potentiometry of the materials comprising the carrier strip. See U.S. Pat. No. 6,743,635 (Neel et al., issued on Jun. 1, 2001) and U.S. Pat. No. 6,946,299 (Neel at al., issued on Sep. 20, 2005), which are herein incorporated by reference. Alternatively, the carrier strip may facilitate the transmission of optical signals which result from differences in the reflection, transmission, scattering, absorption, fluorescence, or electrochemiluminescense of the materials comprising the carrier strip and/or the test pads. See U.S. Pat. No. 6,040,195 (Carroll et al., issued on Mar. 21, 2000) and U.S. Pat. No. 6,284,550 (Carroll et al., issued on Sep. 4, 2001) which are herein incorporated by reference.

The carrier strip's size and shape is only limited by the desired application of the embodiment. For example, if the desired application is testing a human patient, the embodiment, and consequently the carrier strip, may be smaller or larger depending upon the size of the human patient. Likewise, if the desired application involves testing an animal patient, the embodiment, and consequently the carrier strip, may be smaller or larger depending upon the size of the animal patient. In some embodiments, the carrier strip is about 1, about 1.25, about 1.5, about 1.75, about 2, about 2.25, about 2.5, about 2.75, about 3, about 3.25, about 3.5, about 3.75, about 4, about 1-2, about 1-3, about 1-4, about 2-3, about 2-4, or about 3-4 inches in length. The carrier strip's shape may optionally be varied depending upon the desired application of the embodiment. Some applications may require substantially narrow, fat, rectangular, circular, oval, square, triangular, or other shapes, including combinations of the indicated shapes. It is envisioned that the shape of embodiments can be tailored to the shape of the environment in which the embodiments will be applied. Moreover, the carrier strip may contain boundary projections that substantially surround one, two, three, and/or four sides of one or more test pads to collect and/or direct sample application to the one or more test pads. Furthermore, it is envisioned that a handle may be optionally attached to a carrier strip or in contact with a carrier strip, either directly or indirectly.

### Test Reagents and Signaling Reagents

Test reagents and signaling reagents suitable for inclusion in embodiments are well known in the art. Such reagents include, but are not limited to, polyclonal antisera and monoclonal antibodies that have specific binding properties and high affinity for virtually any antigenic substance. Literature affords many means of preparing such reagents. See, e.g., Laboratory Techniques in Biochemistry and Molecular Biology, Tijssen, Vol. 15, Practice and Theory of Enzyme Immunoassays, chapter 13, The immobilization of Immunoreactants on Solid Phases, pp. 297-328, and the references cited therein which are herein incorporated by reference. Additional assay protocols, reagents, and analytes useful in the practice of the invention are known per se. See, e.g., U.S. Pat. No. 4,313,734 (Leuvering, issued on Feb. 2, 1982), columns 4-18, and U.S. Pat. No. 4,366,241 (Tom et al., issued on Dec. 28, 1982), columns 5-40 which are herein incorporated by reference.

Metal sols, including but not limited to gold sol, and other types of colored particles, including but not limited to, organic dye sols and colored latex particles, that are useful as marker substances in immunoassay procedures are also known per se and suitable for use as test reagents and/or signaling reagents. See, for example, U.S. Pat. No. 4,313,734 (Leuvering, issued on Feb. 2, 1982), the disclosure of which is incorporated herein by reference. For details and engineering principles involved in the synthesis of colored particle conjugates see Horisberger, Evaluation of Colloidal Gold as a Cytochromic Marker for Transmission and Scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973) which are herein incorporated by reference.

Test reagents for inclusion in the embodiments may signal directly, such as with an electrical or optical signal (visible either to the naked eye, or with an optical filter or upon applied stimulation to promote fluorescence or phosphorescence). Test reagents may also signal indirectly such as with enzymes, e.g. alkaline phosphatase and/or horseradish peroxidase, in combination with signaling reagents in the form of enzymatic substrates that will generate a signal upon interaction with the enzyme. In some embodiments, the signaling reagent and/or test reagent is incorporated into the test pad. In other embodiments, the signaling reagent and/or test reagent is added to the test sample before application to the test pad. In additional embodiments, the signaling reagent and/or test reagent is added to the test pad after introduction of the test sample.

Alcohol sensitive test reagents and methods are well known in the art. See, e.g. U.S. Pat. No. 5,563,073 (Titmas, issued on Oct. 8, 1996) and Jai Moo Shin et al., Simple Diagnostic Tests to Detect Toxic Alcohol Intoxications, NIH (Oct. 2008), which are hereby incorporated by reference in their entirety. In some embodiments, the test reagent and/or signaling reagent from Alco Screen™ pads, manufactured by Chematics, Inc. located in North Webster, Ind., is incorporated. Optionally, the test reagent and/or signaling reagent from Alco Screen™ pads is incorporated in the one or more test pads, but it may also be applied to the test pad after sample application or it may be applied to the sample before application to the test pad. In some embodiments the test reagent and/or signaling reagent from the alcohol dehydrogenase method (ADH method) is incorporated in the one or more test pads, but it may also be applied to the test pad after sample application or it may be applied to the sample before application to the test pad. In some embodiments the test reagent and/or signaling reagent from the alcohol oxidase method method (ALOx method) is incorporated in the one or more test pads, but it may also be applied to the test pad after sample application or it may be applied to the sample before application to the test pad. In some embodiments the test reagent and/or signaling reagent from the sodium periodate method is incorporated in the one or more test pads, but it may also be applied to the test pad after sample application or it may be applied to the sample before application to the test pad. In some embodiments the test reagent and/or signaling reagent from the potassium permanganate method (PA method) is incorporated in the one or more test pads, but it may also be applied to the test pad after sample application or it may be applied to the sample before application to the test pad.

Test reagents and/or signaling reagents may also detect the storage and handling of embodiments. In some embodiments, test reagents and/or signaling reagents may be sensitive to temperature and if the temperature of the embodiment's environment has exceeded or fallen below a predetermined temperature, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

In some embodiments, test reagents and/or signaling reagents may be sensitive to moisture, and if the humidity of the embodiment's environment has exceeded or fallen below a predetermined level, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

Test reagents and/or signaling reagents may also detect whether a sufficient amount of sample has been applied to an embodiment for analysis. For example, when the sample is saliva, a test reagent and/or signaling reagent specific for a salivary enzyme, such as amylase, may detect the salivary enzyme's presence if a sufficient volume of sample has been applied. The detection of a sufficient sample may optionally be signaled to the user in the form of a color or symbol. Using such embodiments, the user would then know if a sufficient quantity of sample was applied to the one or more test pads to afford an accurate analysis.

Embodiments that detect storage and/or sufficient application of sample volume are particularly capable of reducing the occurrence of false negatives. For example, poor storage conditions may inactivate a test reagent in a test pad. Upon application of sample to such a test pad, no signal may result and a user could believe that an analyte is not present—a false negative. Alternatively, test pads having a pre-printed negative signal may suffer a similar occurrence of a false negative if the test reagent is inactivated because an analytes presence in a sample would not convert the pre-printed negative signal into a positive signal. Likewise, an insufficient volume of sample may generate no signal or a negative signal and cause a user to believe that an analyte is not present.

Any enzyme, antibody, dye buffer, chemical, sol, or combinations thereof may be incorporated so long as the enzyme, antibody, dye buffer, chemical, metal sol, or combinations thereof are capable of detecting the presence of one or more analytes in a sample. See, e.g., U.S. Pat. No. 6,383,736 (Titmas, issued on May 7, 2002), U.S. Pat. No. 7,858,756 (Owens et al., issued on Dec. 28, 2010), and U.S. Pat. No. 7,790,400 (Jehanli et al., issued on Sep. 7, 2010) which are hereby incorporated by reference in their entirety.

### Test Pads

The one or more test pads may be prepared from any bibulous, porous, fibrous, or sorbent material capable of rapidly absorbing a sample. Porous plastics material, such as polypropylene, polyethylene, polyvinylidene flouride, ethylene vinylacetate, acrylonitrile and polytetrafluoroethylene can be used. Optionally, the one or more test pads can be pre-treated with a surface-active agent to reduce any inherent hydrophobicity in the one or more test pads and enhance their ability to absorb a sample. Moreover any one of the one or more test pads may be treated with an oxygen-impermeable water soluble substance. Suitable examples of an oxygen-impermeable water soluble substance include, but are not limited to, polyvinyl alcohol, partly saponified polyvinyl acetate which can also contain vinylether and vinylacetal units, polyvinyl pyrrolidone and copolymers thereof with vinyl acetate and vinyl ethers, hydroxy alkyl cellulose, gelatin, polyacrylic acid, gum arabic, polyacryl amide, dextrin, cyclodextrin, copolymers of alkylvinyl ethers and maleic acid anhydride, ring opened polymers of maleic acid anhydride, water-soluble high molecular polymers of ethylene oxide having molecular weights of above 5,000, and/or polyvinyl alcohol in combination with poly(l-vinylimidazole) or a copolymer of 1-vinyl-imidazole. The one or more test pads can also be made from paper or other cellulosic materials, including but not limited to nitrocellulose. Materials that are now used in the nibs of fiber-tipped pens are also suitable for incorporation in the one or more test pads.

Optionally, the one or more test pads may be prepared from non-porous materials. In such circumstances, the test reagents and/or signaling reagents may be coated on the outer surface of the one or more test pads such that contact with a sample containing an analyte will result in the generation of a signal.

Using known methods, test pads may be shaped or extruded in a variety of lengths and cross-sections. Embodiments may possess one or more test pads of various sizes and shapes, and the size and shape of the one or more test pads are only limited by their number, size, and desired application of the embodiment in which they are incorporated within. In some embodiments, the one or more test pads are substantially similar in size and/or shape. In other embodiments, the one or more test pads may differ substantially in size and/or shape. It is readily envisioned that embodiments may possess about one or more test pads, about two or more test pads, about three or more test pads, about four or more test pads, about five or more test pads, about six or more test pads, about seven or more test pads, about eight or more test pads, about nine or more test pads, about ten or more test pads, about 1-4 test pads, about 1-10 test pads about 1-100 test pads, about 2-100 test pads, about 3-100 test pads, about 4-100 test pads, about 5-100 test pads, about 5-75 test pads, about 10-50 test pads, about 15-25 test pads, and individual numbers of test pads therein. The one or more test pads may be made of the same material, or optionally they may be made of different materials or even combinations of different materials. Moreover, the one or more test pads may be recessed into the carrier strip.

In some embodiments, test pads may be prepared from a single layer of material. In other embodiments, test pads may be prepared from multiple layers of material. It is readily envisioned that embodiments may possess about one or more layers, about two or more layers, about three or more layers, about four or more layers, about five or more layers, about six or more layers, about seven or more layers, about eight or more layers, about nine or more layers, about ten or more layers, about 1-4 layers, about 1-5 layers, about 1-6 layers, about 1-7 layers, about 1-8 layers, about 1-9 layers, about 1-10 layers, about 1-100 layers, about 2-100 layers, about 3-100 layers, about 4-100 layers, about 5-100 layers, about 5-75 layers, about 10-50 layers, about 15-25 layers, and individual numbers of layers therein.

The test pad layers may be of the same or different materials. Test reagents and/or signaling reagents may also be impregnated in a single layer of material or in multiple layers of material. The impregnation may take any suitable form, including, but not limited to, a substantially uniform impregnation or impregnation with dots or stripes. Test reagents and/or signaling reagents can be impregnated in various concentrations in one or more of the multiple layers to tailor the sensitivity of the test pads to certain analytes. Such sensitivity could afford information about the concentration of an analyte in the sample. Furthermore, the impregnation may optionally be conducted in a manner that will generate a signal observable by the user upon application of a sufficient quantity of sample, detection of an analyte, or proper/improper storage of the embodiment.

When one or more test pads are comprised of multiple layers of material, one or more layers of material may be impregnated (e.g. pre-printed) with an inert chemical such that a line or “minus sign” is displayed to the user. In some embodiments, the line or “minus sign” could be in the form of a material covering the one or more test pads to give a visual impression of a line or “minus sign” on the one or more test pads. One or more additional layers of the material comprising the one or more test pads could then be impregnated with a test reagent and/or a signaling reagent that upon detecting a sufficient quantity of sample, appropriate storage temperature, and/or the presence of an analyte, the impregnated test reagent and/or signaling reagent will create a perpendicular line such that a “plus sign” will be signaled to the user. In other embodiments, the line or “minus sign” displayed in the one or more test pads could be obscured by color or opaqueness when a test reagent and/or a signaling reagent detects a sufficient quantity of sample, appropriate or inappropriate storage temperature, and/or the presence of an analyte.

The test pad layers may comprise optically transparent membranes. Detection on an analyte may then generate a signal that is opaque, partially transparent, or completely transparent. Moreover, test pad layers may be only partially optically transparent prior to application of a sample. Alternatively, the application of a sample to one or more test pad layers may result in the layers becoming optically transparent, thereby allowing a user to see generated and/or pre-printed signals on test pad layers below the optically transparent layers. Moreover, the individual layers in a test pad may be positioned such that the detection of an analyte in a lower layer of material is obscured by the detection of an analyte in a layer of material positioned above the lower layer.

It is also envisioned that embodiments may have arrangements of test pads and/or arrangements of layers within multiple layered test pads such that the detection of an analyte in the test pads or the layers of a test pad generate a signal, such as a “plus sign” or “minus sign” to the user. Such embodiments may comprise at least two layers of material, each capable of generating a line upon detecting an analyte or a certain concentration of an analyte. Optionally, the lines may intersect to generate a “plus” sign or other signal upon the detection of an analyte in the at least two layers of material. Alternatively, embodiments may comprise at least four layers of material, each capable of generating a line upon detecting an analyte or a certain concentration of an analyte in the at least four layers of material. Optionally, the lines may intersect at one or more points such that a “plus” sign or other symbol is formed. While the aforementioned embodiments have been discussed with reference to “minus” and “plus” signs, it is envisioned that any symbol, including color changes, could be used to convey similar information to a user. Such symbols include, but are not limited to, circles, ovals, squares, triangles, trapezoids, rhombi, plus signs, minus signs, “X” shaped signs, checkmarks, and/or dotted, dashed, or differentially colored version of said symbols. The meaning of any desired symbol or color change could be included in the packaging of an embodiment or imprinted on an embodiment.

The test reagents applied to each layer of material may optionally be the same or different. When different test reagents are applied to different layers of material comprising the one or more test pads, the test pad may be tailored to generate a signal indicating the diagnosis of one or more illnesses, diseases, or injuries. One method for achieving such a diagnosis would be to have the individual layers comprising the test pad generate a signal in response to one or more symptoms of one or more illnesses, diseases, or injuries. For example, if the diagnosis of one or more illnesses, diseases, or injuries required the determination of multiple analytes, then the detection of each analyte could produce a portion of a symbol that is visible to the user. Upon formation of a complete symbol, the embodiment would confirm the presence of a certain illness, disease, or injury. Optionally, information relating to each specific analyte could be transferred to the one or more memory devices.

One can readily appreciate the application of such embodiments of multiple layer test pads when knowledge of a certain concentration is needed. As a non-limiting application, the detection of a person's blood alcohol level may be achieved using such an embodiment. For a test pad comprising at least four test pad layers, if a first test pad layer was sensitive to a blood alcohol level of at least 0.02%, a second test pad layer was sensitive to a blood alcohol level of at least 0.04%, a third test pad layer was sensitive to a blood alcohol level of at least 0.06%, and a fourth test pad layer was sensitive to a blood alcohol level of at least 0.08%, then the application of a sample having a blood alcohol level at least at the sensitive percentages would generate a signal. Assuming that operating a motor vehicle with a blood alcohol level equal to or greater than 0.08% is illegal, then the application of a sample that generates a “plus” sign would indicate that the sample provider should not legally operate a motor vehicle. One will readily appreciate that this described example is capable of extension to any number of test pads having any number of layers, such that the detection of an analyte in each layer generates a signal indicative of concentration.

As another non-limiting example, test reagents and/or signaling reagents that are sensitive to markers specific for hepatitis and/or liver damage may be applied to test pads and/or layers within test pads. Consequently, the detection of markers specific for hepatitis and/or liver damage in each test pad and/or layers within test pads would generate a signal. An individual test pad may optionally be sensitive to a single marker for hepatitis and/or liver damage. Alternatively, a single test pad may be sensitive to multiple markers for hepatitis and/or liver damage. In such an embodiment, the detection of one or more markers for hepatitis and/or liver damage may produce a certain signal, e.g. color, indicative of the number of markers detected and/or indicative of the exact marker detected. Alternatively, an embodiment may produce a signal in the form of a shape that indicates the presence of one or more markers indicative of hepatitis and/or liver damage. For example, an embodiment may have a test pad with four or more test pad layers, while each layer may be sensitive to one or more markers specific to an analyte such as viral hepatitis. The respective detection of a marker in each of the test pad would generate a signal such that the detection of a marker in each of the test pad layers would confirm the diagnosis of a viral hepatitis. Although such an embodiment has been described with specific references to a viral hepatitis, it is envisioned that such an embodiment may readily be tailored to detect any number of analytes and/or markers that are specific to any analyte described below.

Embodiments may optionally possess one or more test pads and test reagents that detect analytes important to a certain age population (e.g. infants, children, young adults, adults, or elderly individuals). It is also envisioned that embodiments could possess one or more test pads and test reagents that detect analytes important to certain categories of individuals (e.g., law enforcement agents, government employers, military members, chronic drug users, physicians, veterinarians, dentists, parents, private sector employers, aid workers, inmates, hospital patients, nursing home patients, outdoorsmen, immuno-compromised individuals, or students). Embodiments may also be directed to analytes important to geographic regions (e.g. third-world countries, developed countries, or specific climate regions). Such embodiments of the invention simplify the number of different embodiments that a user must purchase or travel with because users can select embodiments that will detect the analytes the users are most interested in, or are most pertinent to a user's current or impending circumstances.

In one embodiment, a single test pad contains or has applied to it a single test reagent and/or signaling reagent suitable for detecting a single analyte. In another embodiment, two or more test pads contain or have applied to one or more of them a single test reagent and/or signaling reagent suitable for detecting a single analyte. Optionally, the single test reagent and/or signaling reagent on or applied to the two or more test pads may be the same or different. Furthermore, when different test reagents and/or signaling reagents are used, the test reagents may be sensitive to the same marker on an analyte or the test reagents may be sensitive to different markers on an analyte. The analyte may optionally be the same or different. When different analytes and different test reagents and/or signaling reagents are used, the analytes and test reagent and/or signaling reagents may be tailored to detect different symptoms of the same illness, disease, or injury. In some embodiments, a diagnosis can be made based upon the detection of all the symptoms specific to an illness, disease, or injury. In other embodiments, a diagnosis can be made based upon the absence of one or more analytes specific to an illness, disease, or injury. Using these described test pads, it is readily apparent that the reduction of false negatives and false positives can be achieved by including redundancy in the embodiments.

In one embodiment, a single test pad may contain or have applied to it two or more reagents suitable for detecting and/or signaling a single analyte. These two or more test reagents and/or signaling reagents may be sensitive to the same marker of an analyte. Optionally, these two or more reagents may be sensitive to different markers on the same analyte. In some embodiments, the two or more test reagents and/or signaling reagents may be applied to the same region of the test pad. In other embodiments, the two or more test reagents and/or signaling reagents may be applied to different regions of the same test pad. The number of test reagents and/or signaling reagents suitable for incorporation or application to a single test pad is limited only by the application of the diagnostic test strip. It is readily envisioned that embodiments may possess about one or more, about two or more, about three or more, about four or more, about five or more, about six or more, about seven or more, about eight or more, about nine or more, about ten or more, about 1-4, about 1-10, about 1-100, about 2-100, about 3-100, about 4-100, about 5-100, about 5-75, about 10-50, about 15-25, and individual numbers therein, of test reagents and/or signaling reagents incorporated or applied to one or more test pads. Using these described test pads, it is readily apparent that the reduction of false negatives and false positives can be achieved by including redundancy in the embodiments.

The one or more test pads suitable for use in an embodiment will readily detect analytes present in liquid samples, such as saliva. It is also envisioned that a test pad may be capable of detecting an analyte present in solid and/or semi-solid samples. When solid and/or semi-solid samples are analyzed, it is understood that a liquid may optionally be applied to the test pad to facilitate analysis.

When liquids and/or liquid samples are applied to test pads, lateral flow through material may result from surface tension, cohesion, adhesion, wicking, and/or capillary action. In general, embodiments that utilize lateral flow will require substantial amounts of a liquid sample for sufficient contacting of the sample with a devices test area. In some embodiments, lateral flow is confined to the test pad region. In other embodiments, lateral flow is confined to individual test pads. In further embodiments, lateral flow is confined to individual layers of a multi-layer test pad. Moreover, some embodiments overcome the use of lateral flow by having a test pad designed to absorb the fluid sample without requiring surface tension, cohesion, adhesion, wicking, and/or capillary action to contact the fluid sample with the test area. Such embodiments are particularly suited for use when the volume of a fluid sample is small and/or limited. This includes, but is not limited to, instances when the fluid sample is oral fluid such as saliva.

### Analytes

An assay based on the principles described herein can be used to determine a wide variety of analytes by choice of appropriate test reagents and/or signaling reagents. The embodiments described herein can be used to test for the existence of analytes including, but not limited to, drugs, especially drugs of abuse; heavy metals; pesticides; pollutants; proteins; polynucleotides such as DNA, RNA, rRNA, tRNA, mRNA, and siRNA; hormones; vitamins; microorganisms such as bacteria, fungi, algae, protozoa, multi-cellular parasites, and viruses; tumor markers; liver function markers; kidney function markers; blood coagulation factors; and toxins. The embodiments may also optionally detect metabolites of each of the aforementioned examples of analytes. Furthermore, some embodiments may also detect their storage conditions, specifically the temperature and humidity of their environment, and/or the application of an appropriate quantity of sample for analysis.

Analytes may be reference analytes or target analytes. Any given analyte may be either a reference analyte or a target analyte, depending upon the desired application. Indeed, any analyte described below that is known to consistently be present in a given sample may serve as a reference analyte. As a non-limiting example, alpha-amylase is an enzyme present in saliva and could serve as a reference analyte when the analyzed sample is saliva. However, methadone could serve as a reference analyte when an embodiment is desired for use with samples obtained from patients generally known and/or suspected of having methadone in their system. Thus, one will readily appreciate that it is the application of the embodiment that determines the analytes classified as references or targets.

More specific examples of drug analytes, including both drugs of abuse and therapeutic drugs, include opiates, which includes but is not limited to methadone, morphine, heroin, dextromethorphen, meperidine, codeine, hydromorphone, pholcodine, and metabolites thereof.

More specific examples of drug analytes, including both drugs of abuse and therapeutic drugs, include benzheterocyclics, the heterocyclic rings being azepines, diazepines and phenothiazines. Examples of azepines include fenoldopam. Examples of benzodiazepines include alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordiazepam, N-Desmethyld, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine. Examples of other benzheterocyclics include, but are not limited to, carbamazepine and imipramine.

Additional drug analytes, including both drugs of abuse and therapeutic drugs, include alkaloids, such as agents that interact with opioid receptors including morphine, dihydromorphine, desomorphine, hydromorphone, nicomorphine, oxymorphone, hydromorphinol, nalbuphine, naloxone, naltrexone, buprenorphine, etorphine, metopon, diacetyldihydromorphine, thebacon, methodone, codeine, hydrocodone, dihydrocodeine, oxycodone, papaveretum, oripavine, thebaine, tapentadol, and heroin; agents that exert effects on serotonin receptors, such as cocaine (and other reuptake inhibitors, including norepinephrine, dopamine, and serotonin reuptake inhibitors); cocaine metabolites such as benzoylecgonine; ergot alkaloids; steroid alkaloids; iminazoyl alkaloids; quinazoline alkaloids; isoquinoline alkaloids; quinoline alkaloids; and diterpene alkaloids.

Another group of drug analytes, including both drugs of abuse and therapeutic drugs, includes steroids, including the estrogens, gestogens, androgens, andrenocortical steroids, bile acids, cardiotonic glycosides and aglycones, which includes digoxin and digoxigenin, saponins and sapogenins, their derivatives and metabolites.

Additional drug analytes, including both drugs of abuse and therapeutic drugs, is the barbiturates, such as barbital, allobarbital, amobarbital, aprobarbital, alphenal, brallobarbital, hexobarbital, Phenobarbital, phencyclidine (PCP), pentobarbital, Nembutal, secobarbital, diphenylhydantonin, primidone, and ethosuximide. Additionally, drugs similar in effect to barbiturates are potential analytes, such as methaqualone, cloroqualone, diproqualone, etaqualone, mebroqualone, mecloqualone, methylmethaqualone, and nitromethaqualone.

Another group of drug analytes, including both drugs of abuse and therapeutic drugs, is aminoalkylbenzenes, including the phenethylamines such as amphetamine, methamphetamine, ephedrine, amphepramone, prolintane, lisdexamfetamine, mescaline, and catecholamines, which includes ephedrine, L-dopa, epinephrine, narceine, and papaverine.

Additional drug analytes, including both drugs of abuse and therapeutic drugs, includes those derived from marijuana, which includes cannabinol, tetrahydrocannabinol, 11-nor-9-carboxy-delta-9-tetrahydrocannabinol (THC), nabilone, dronabinol, marinol, and cannabinoids such as cannabidiol, cannabinol, and tetrahydrocannabivarin.

Another group of drug analytes, including both drugs of abuse and therapeutic drugs, are those that interact with the N-methyl d-aspartate (“NMDA”) receptor, including agonists, modulators, and antagonists such as 1-(1-phylcyclohexyl)piperidine (phencyclidine or “PCP”), R-2-amino-5-phosphonopentanoate, 2-amino-7-phosphonoheptanoic acid, (3-[(R)-2-carboxypiperazin-4-yl]-prop-2-enyl-1-phosphonic acid), PEAQX, selfotel, amantadine, dextrallorphan, dextromethorphan, dextrorphan, dizocilpine, ethanol, eticyclidine, gacyclidine, ibogaine, ketamine, memantine, methoxetamine, rolicyclidine, tenocyclidine, tiletamine, neramexane, eliprodil, etoxadrol, dexoxadrol, NEFA, remacemide, delucemine, 8A-PDHQ, aptiganel, HU-211, remacemide, atomoxetine, rhynchophylline, 1-aminocyclopropanecarboxylic acid, 7-chlorokynurenate, 5,7-dichlorokynurenic acid, kynurenic acid, and lacosamide.

Another group of therapeutic drugs is antibiotics, which include, for example, beta-lactam antiobiotics such as penicillins and cephalosporins, penems and carbapenems, antimicrobials such as aminoglycosides, ansamycins, carbacephems, glycopeptides, lincosamides, lipopetides, macrolides, monobactams, nitrofurans, quionolones, polypeptide-based antibiotics, chloromycetin, actinomycetin, spectinomycin, sulphonamides, trimethoprim, tetracyclines, and beta-lactamase inhibitors such as calvulanic acid, tazobactam, and sulbactam.

Other individual miscellaneous drug analytes, including both drugs of abuse and therapeutic drugs, include nicotine, caffeine, cotinine, gamma-hydroxybutyric acid, dextromoramide, ketobemidone, piritramide, dipipanone, phenadoxone, benzylmorphine, codeine, nicocodeine, dihydrocodeinone enol acetate, tilidine, meptazinol, propiram, acetyldihydrocodeine, pholcodine, 3,4-methylenedioxymethamphetamine, psilocybin, 5-methoxy-N,N-diisopropyltryptamine, peyote, 2,5-dimethoxy-4-methylamphetamine, 2C-T-7 (a psychotropic entheogen), 2C—B, cathinone, alpha-methyltryptamine, bufotenin, benzylpiperazine, methylphenidate, dexmethylphenidate, laudanum, fentanyl, mixed amphetamine salts (i.e. Adderall), lisdexamfetamine, dextroamphetamine, dextromethamphetamine, phentermine, phylpropanolamine, ephedrine, pethidine, anabolic steroids, talbutal, butalbital, buprenorphine, xyrem, paregoric, modafinil, difenoxin, diphenoxylate, promethazine, pregabaline, pyrovalerone, atropine, and other Schedule I-V classified drugs, glucose, cholesterol, bile acids, fructosamine, carbohydrates, metals which includes, but is not limited to lead and arsenic, alcohols (i.e. methanol, ethanol, propanol, butanol, ethylene glycol, diethylene glycol, C₅₋₁₀ containing alcohols, and metabolites thereof), meprobamate, serotonin, meperidine, amitriptyline, nortriptyline, lidocaine, procaineamide, acetylprocainearnide, propranolol, griseofulvin, valproic acid, butyrophenones, antihistamines, and anticholinergic drugs, such as atropine.

Pesticide analytes of interest include categories such as algicides, avicides, bactericides, fungicides, herbicides, insecticides, miticides, molluscicides, nematicides, rodenticides, virucides, and specifically polyhalogenated biphenyls, phosphate esters, thiophosphates, carbamates, and polyhalogenated sulfenamides.

Additional chemical analytes of interest include fertilizers such as ammonium derivatives, nitrates, and phosphates; heavy metals such as lead, mercury, uranium, plutonium, arsenic, cadmium, chromium, and nickel

More specific examples of protein analytes include antibodies, protamines, histones, albumins, globulins, scleroproteins, phosphoproteins, mucoproteins, chromoproteins, lipoproteins, nucleoproteins, glycoproteins, proteoglycans, and unclassified proteins, such as somatotropin, prolactin, insulin, and pepsin. A number of proteins found in the human plasma are important clinically and include prealbumin, albumin, α₁-lipoprotein, α₁-acid glycoprotein, α₁-antitrypsin, α₁-glycoprotein, transcortin, 4.6S-postalbumin, tryptophan-poor, α₁-glycoprotein, α₁X-glycoprotein, thyroxin-binding globulin, inter-α-trypsin-inhibitor, Gc-globulin (Gc I-I, Gc 2-1, Gc 2-2), haptoglobin, ceruloplasmin, cholinesterase, α₂-lipoprotein(s), myoglobin, C-reactive Protein, α₂-macroglobulin, α₂-HS-glycoprotein, Zn-α₂-glycoprotein, α₂-neuramino-glycoprotein, erythropoietin, β-lipoprotein, transferrin, hemopexin, fibrinogen, plasminogen, β₂-glycoprotein I, β₂-glycoprotein II, immunoglobulins A, D, E, G, M, prothrombin, thrombin, and protein markers in cancers including, but not limited to, breast cancer, prostate cancer, melanoma, carcinoma, pancreatic cancer, liver cancer, and brain cancer.

Additional protein analytes of interest include alanine aminotransferase and aspartate aminotransferase. Alanine aminotransferase is markedly elevated when hepatitis is present in the liver. Such elevation for alanine aminotransferase may include at least about 1.25, 1.5, 1.75, 2, 2.25, 2.5, 2.75, and 3.0 times the normal levels associated with a person lacking liver damage. Aspartate aminotransferase is elevated when cellular damage occurs, such as liver damage, skeletal muscle damage, and acute myocardial infarction. Additionally, levels are elevated because of congestive heart failure, pericarditis, cirrhosis, metastatic liver disease, skeletal muscle diseases, and generalized infections such as mononucleosis. Such elevation for aspartate aminotransferase may include at least about 1.25, 1.5, 1.75, 2, 2.25, 2.5, 2.75, and 3.0 times the normal levels associated with a person lacking liver damage. Consequently, the detection of alanine aminotransferase and/or aspartate aminotransferase is of therapeutic importance.

Specific examples of peptide and protein hormone analytes include parathyroid hormone (parathromone), thyrocalcitonin, insulin, glucagon, relaxin, erythropoietin, melanotropin (melanocyte-stimulating hormone and intermedin), somatotropin (growth hormone), corticotropin (adrenocorticotropic hormone), thyrotropin, prolactin, follicle-stimulating hormone, luteinizing hormone), chorionic gonadotropin (hCG), oxytocin, and vasopressin.

Specific examples of polynucleotide analytes include DNA and RNA as well as their nucleoside and nucleotide precursors, which include ATP, NAD, FMN, adenosine, guanosine, thymidine, cytidine, and uracil with their appropriate sugar and phosphate substituents.

Specific examples of vitamin analytes include Vitamin A (i.e. retinol), B (e.g. B₁ or thiamine, B₂ or riboflavin, B₃ or niacin, B₅ or pantothenate, B₆ or pyridoxine, B₇ or biotin, B₉ or folic acid, and B₁₂), C (i.e. ascorbic acid), D (e.g. calciferol, D₂, and D₃), E (i.e. tocopherol), K, and vitamin derivatives or metabolites such as nicotinamide.

Specific examples of microorganism analytes, including infectious disease agents, include corynebacteria, pneumococci, streptococci, staphylococci, neisseriae, hemophilus influenzae, pasteurellae, brucellae, aerobic spore-forming bacilli, anaerobic spore-forming bacilli, mycobacteria, actinomycetes (fungus-like bacteria), the spirochetes, mycoplasmas, and other pathogens, such as listeria monocytogenes, erysipelothrix rhusiopathiae, streptobacillus moniliformis, donvania granulomatis, bartonella bacilliformis, rickettsiae (bacteria-like parasites), fungi, agents causing venereal diseases such as chlamydia, chancroid, granuloma inguinale, gonorrhea, syphilis, jock itch, yeast infection, herpes simplex, HPV, crab louse, scabies, trichomoniasis, and infectious diarrheal microorganisms such as camplylobacter, salmonellae, shigellae, Escherichia coli, Clostridium difficile, Giardia lamblia, Entamoeba histolytica, and organisms causing leptospirosis, nosocomial infections, staphylococcal enterotoxicosis, typhoid fever, cholera, vibrio gastroenteritis, yersinia gastroenteritis, clostridium perfringens gastroenteritis, bacillus cereus gastroenteritis, aflatoxin poisoning, amoebic dysentery, cryptosporidiosis, cyclospora diarrheal infection. Other microorganism analytes include viruses, such as herpes viruses, pox viruses, picornaviruses, myxoviruses (influenza A, B, and C, and mumps, measles, rubella, etc.), arboviruses, reoviruses, rotoviruses, noroviruses, adenoviruses, astroviruses, hepatitis, human immunodeficiency virus, and tumor viruses.

The categories of protein analytes and microorganism analytes may optionally overlap. For example, a microorganism analyte may be detected via the analysis of a protein analyte specific for the microorganism analyte. A protein analyte specific for a microorganism analyte may include an antibody specific for a microorganism analyte, or marker thereof. As a non-limiting example, for a microorganism analyte such as viral hepatitis, antibodies specific to any of viral hepatitis A, B, C, D, E, F and/or G may comprise the protein analyte. Such antibodies include, but are not limited to, immunoglobins such as IgA, IgD, IgE, and specifically IgM and/or IgG, and antibodies to surface antigens, envelope antigens, core antigens, and/or delta antigens (e.g. small and/or large). Specific examples of antigens for viral hepatitis B include hepatitis B surface antigen (HBsAg), hepatitis B envelope antigen (HBeAg), hepatitis B core antigen (HBcAg). Alternatively, a protein analyte specific for a microorganism analyte may include a protein analyte characteristically produced by the microorganism analyte. As a non-limiting example, for a microorganism analyte such as viral hepatitis, proteins specific to any of viral hepatitis A, B, C, D, E, and/or F may comprise the protein analyte. Such protein analytes include, but are not limited to, structural and/or nonstructural proteins. Specific examples of protein analytes for viral hepatitis C include, but are not limited to structural proteins such as E1 and/or E2, and/or nonstructural proteins such as NS2, NS3, NS4, NS4A, NS4B, NS5, NS5A, NS5B, and peptide portions thereof.

The above described analytes possess at least one marker recognized by at least one test reagent and/or signaling reagent. Optionally, the above described analytes may possess multiple markers recognized by the same and/or different test reagents and/or signaling reagents. It is readily envisioned that a marker may be the entire analyte and/or a portion thereof.

A preferred embodiment are testing for analytes detectable through saliva. It is beneficial to test for analytes to aid in the detection of drugs of abuse and thereapeutic drugs, as well as cancer markers, disease markers, hormonal markers, glucose and metabolites.

More specific examples of salivary drug analytes for detecting both drugs of abuse and therapeutic drugs are ethanol, methanol, ethylene glycol, and diethylene glycol.

More specific examples of salivary drug analytes, including both drugs of abuse and therapeutic drugs, include opiates, which includes but is not limited to methadone, morphine, 6-monoacetytl morphine, heroin, dextromethorphen, meperidine, codeine, cocaine, hydromorphone, pholcodine, and metabolites thereof.

Additional salivary drug analytes, including both drugs of abuse and therapeutic drugs, is the barbiturates, such as barbital, amobarbital, hexobarbital, Phenobarbital, methyl phenobarbital, phencyclidine (PCP), pentobarbital.

Additional salivary drug analytes, including both drugs of abuse and therapeutic drugs, includes those derived from marijuana, which includes cannabinol, tetrahydrocannabinol, 11-nor-9-carboxy-delta-9-tetrahydrocannabinol (THC).

More specific examples of salivary drug analytes, including both drugs of abuse and therapeutic drugs, include benzodiazepines including alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordiazepam, N-Desmethyldiazepam, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine.

Other individual miscellaneous salivary drug analytes, including both drugs of abuse and therapeutic drugs, include nicotine and cotinine.

Specific examples of salivary analytes used for detecting cancer include mRNA biomarkers for pancreatic cancer, mRNA markers for oral cancer, HER2/neu, CA 15-3, p53, transferrin, cyclin D1, and maspin (serpin B5).

Specific examples of salivary analytes used for detecting certain metabolic disorders include glucose, anti-HIV antibody, HBV surface antigen, anti-HAV (IgM and IgG), anti-Helicobacter pylori (anti-H. pylori IgG), allergen-specific IgA, chromogranin A, lysozyme, peroxidase, hydroxyproline, calcium, and C-reactive protein.

Specific examples of salivary analytes used for detecting hormones include cortisol, alpha amylase, estradiol, progesterone, dehydroepiandrosterone (DHEA), testerosterone, leutinizing hormone, melatonin, and cyclin D.

Specific examples of salivary analytes used for detecting and investigating human psychological phenomenon are cortisol and alpha amylase.

### Samples

An analyte of interest may be present in a wide variety of environments, and it is envisioned that a person having ordinary skill in the art will readily understand that the components and embodiments discussed above can be modified as needed to accommodate different environments of samples.

Analytes of interest may be found in a patient's physiological fluids, such as mucus, blood, serum, blood plasma, lymph, puss, urine, feces, cerebral spinal fluid, ocular lens liquid, ascites, semen, sputum, saliva, sweat, and secreted oils. Samples for testing analytes may be obtained using techniques known or envisioned to provide samples of such physiological fluids. Optionally, analytes may be detected by directly contacting embodiments of the diagnostic test strips with the patient's body, such as their skin, eyes, mouth cavity regions including the tongue, tonsils, and inner lining of the mouth and throat, and the nasal cavity. Alternatively, some analytes may be detected by directly contacting embodiments of the diagnostic test strips with a patient's urine stream, source of bleeding, source of puss, discharge from sex organs, or other site of fluid leakage from the patient.

Analytes may also be found in synthetic chemicals, water, soil, air and food (e.g., milk, meat, poultry, or fish). Any organic- and inorganic-containing substances can serve as an analyte so long as test reagents are available to generate a signal concerning the presence, absence, and/or concentration of the analyte.

For oral fluids such as saliva, samples may be obtained by contacting an embodiment with a patient's tongue such that the tongue contacts the one or more test pads. Alternatively, salivary samples may be obtained by contacting an embodiment with the top and/or sides of a patient's tongue using a substantially back and forth motion from substantially the tip of the tongue to substantially the back of the tongue. Furthermore, salivary samples may be obtained by contacting an embodiment with the top and/or sides of a patient's tongue using a substantially side-to-side motion along the width of the tongue. Similarly, salivary samples may also be obtained by contacting an embodiment with the top and/or sides of a patient's tongue using a substantially circular motion. For each of the above described sample collection methods, the results of the analysis could then be read directly from the diagnostic test strip by a user. Optionally, test results could be stored to a suitable memory device for recordation and later access.

Prior to use with embodiments of the invention, samples may be preserved, stored, or pre-treated in manners consistent with known handling of the same, or similar, types of samples. It is envisioned that any type of preservation, storage, or pre-treatment may be utilized so long as it does not introduce false positives or false negatives into the assay.

### CONCLUSION

While the invention has been described with reference to the specific embodiments thereof, it should be understood by those skilled in the art that various changes may be made and equivalents may be substituted without departing from the true spirit and scope of the invention. For example, some embodiments do not provide all of the benefits and features set forth herein. In addition, many modifications may be made to adapt a particular situation, material, composition of matter, process, process step or steps, to the objective, spirit and scope of the present invention. Furthermore, practiced embodiments may include features of more than one of the described embodiments. All such modifications are intended to be within the scope of the claims appended hereto. Accordingly, the scope of the invention is defined only by reference to the appended claims.

What is claimed is:

1. A diagnostic test strip for detecting analytes wherein said strip has an active reference zone, comprising:
a) a carrier strip; and
b) at least one test pad further comprising:
1) a first transparent membrane containing a test reagent that indicates the presence of at least one reference analyte; and
2) a second transparent membrane containing a test reagent that indicates the presence of at least one target analyte;
wherein each of the test reagents are arranged in a substantially single striped shape on a portion of the transparent membranes, and the transparent membranes are opposed to each other such that the striped shapes are at substantially right angles, and the at least one test pad is in fluid contact with the carrier strip.

2. The diagnostic test strip of claim 1, wherein there is a single test pad.

3. The diagnostic test strip of claim 1, wherein there is at least two or more test pads.

4. The diagnostic test strip of claim 3, wherein the at least two or more test pads detect different target analytes.

5. The diagnostic test strip of claim 3, wherein the at least two or more test pads detect different markers on the same analyte.

6. The diagnostic test strip of claim 3, wherein the at least two or more test pads are separated on the carrier strip.

7. The diagnostic test strip of claim 3, wherein the at least two or more test pads are in direct fluid contact with each other.

8. The diagnostic test strip of claim 3, wherein at least two of the two or more test pads are on opposite sides of the carrier strip.

9. The diagnostic test strip of claim 1, wherein the second transparent membrane contains two or more separate test reagents for target analytes each at substantially right angles to the test reagent for the reference analyte on the first membrane.

10. The diagnostic test strip of claim 1, wherein the at least one test pad is substantially covered in an oxygen-impermeable water-soluble membrane.

11. The diagnostic test strip of claim 1, wherein the reference analyte and the target analyte are found in saliva.

12. The diagnostic test strip of claim 1, wherein the reference analyte is alpha-amylase.

13. The diagnostic test strip of claim 1, wherein the target analyte is a drug of abuse.

14. The diagnostic test strip of claim 1, wherein the target analyte is a therapeutic drug.

15. The diagnostic test strip of claim 1, wherein the reference analyte and the target analyte are found in sputum.

16. The diagnostic test strip of claim 1, wherein the reference analyte and the target analyte are found in blood serum.

17. The diagnostic test strip of claim 1, wherein the reference analyte and the target analyte are found in blood plasma.

18. The diagnostic test strip of claim 1, wherein the reference analyte and the target analyte are found in blood.

19. The diagnostic test strip of claim 1, wherein the reference analyte and the target analyte are found in urine.

20. The diagnostic test strip of claim 1, wherein the reference analyte and the target analyte are found in semen.

21. The diagnostic test strip of claim 1, wherein the reference analyte and the target analyte are found in ascites.

22. The diagnostic test strip of claim 1, wherein the reference analyte and the target analyte are found in cerebral spinal fluid.

23. The diagnostic test strip of claim 1, wherein the reference analyte and the target analyte are found in fecal matter.

24. The test strip of claim 1, wherein the carrier strip further comprises an element mechanically fixing the test pad to the carrier strip.

25. A method for detecting one or more analytes in a patient sample, comprising:
a) contacting the test strip of claim 1 with a patient sample so that the sample contacts the one or more test pads; and
b) reading the results from the test strip.

26. The method of claim 25, further comprising contacting the test strip with one or more signaling reagents so that the one or more reagents contact the one or more test pads.

27. The method of claim 25, wherein the patient sample is serum.

28. The method of claim 25, wherein the patient sample is semen.

29. The method of claim 25, wherein the patient sample is urine.

30. The method of claim 29, wherein the test strip is directly contacted with the patient's urine stream.

31. The method of claim 25, wherein the patient sample is saliva.

32. The method of claim 31, wherein the test strip is contacted with patient's tongue.

33. The method of claim 25, wherein the patient sample is blood.

34. The method of claim 33, wherein the test strip is contacted directly with the source of the blood.

35. The method of claim 25, wherein the patient sample is ascites.

36. The method of claim 25, wherein the patient sample is sputum.

37. The method of claim 25, wherein the patient sample is cerebral spinal fluid.

38. The method of claim 25, wherein the patient sample is fecal matter.

39. A diagnostic test strip for detecting analytes wherein said strip has an active reference zone, comprising:
a) a carrier strip;
b) at least one test pad further comprising:
1) a first transparent membrane containing a test reagent that indicates the presence of at least one reference analyte;
2) a second transparent membrane containing a test reagent that indicates the presence of at least one target analyte;
wherein each of the test reagents are arranged in a substantially single striped shape on a portion of the transparent membranes, and the transparent membranes are opposed to each other such that detecting both the reference analyte and the target analyte produces a signal in the shape of a checkmark.

40. A diagnostic test strip for detecting analytes wherein said strip has an active reference zone, comprising:
a) a carrier strip;
b) at least one test pad further comprising:
1) a first transparent membrane containing a test reagent that indicates the presence of at least one reference analyte;
2) a second transparent membrane containing a test reagent that indicates the presence of at least one target analyte;
wherein the membranes are opposed and have their test reagents arranged such that a signal produced upon detection of an analyte by a test reagent in one membrane is contained within a signal produced upon the detection of an analyte by a test reagent in another membrane.

41. The diagnostic test strip of claim 40, wherein the signal generated by a test reagent in one membrane is a circle, oval, or square and the signal generated by a test reagent in another membrane is a plus sign, and “X” sign, or a checkmark.
